(19) United States
(12) Patent Application Publication
(10) Pub. No.: US 2025/0049310 A1
(43) Pub. Date: Feb. 13, 2025

(54) ENDOSCOPIC TUBE SET

(71) Applicant: HUKUI BIOTECHNOLOGY CO., LTD., Taipei City (TW)

(72) Inventors: SHIH-CHE LO, Taipei City (TW); YI-MING CHU, Taipei City (TW); MING-KUN CHAN, Taipei City (TW)

(73) Assignee: HUKUI BIOTECHNOLOGY CO., LTD., Taipei City (TW)

(21) Appl. No.: 18/795,216

(22) Filed: Aug. 6, 2024

Related U.S. Application Data
(60) Provisional application No. 63/517,934, filed on Aug. 7, 2023.
(60) Provisional application No. 63/589,998, filed on Oct. 13, 2023.

(30) Foreign Application Priority Data
Jun. 6, 2024 (TW) 113121049

(51) Int. Cl. A61B 1/018; A61B 1/00; A61B 1/005; A61B 1/12

(52) U.S. Cl. CPC A61B 1/018; A61B 1/00082; A61B 1/0052; A61B 1/0057; A61B 1/009; A61B 1/126

(57) ABSTRACT

An endoscopic tube set includes an endoscopic tube including a body; a catheter connected to the body, having therein one single working channel, having a proximal opening proximal to the body, and having a distal opening distal to the body; and a position limiting component fitted to the distal opening of the catheter, having a position limiting opening not round in shape, and having a position limiting mark; and a working element having a cross section corresponding in shape to the position limiting opening of the position limiting component and having at least one positioning mark. The endoscopic tube set is suitable for observing, sampling and/or treating pulmonary lesions.

### CROSS-REFERENCE TO RELATED APPLICATION

This non-provisional application claims priority under 35 U.S.C. § 119(a) on Patent Application No(s).113121049 filed in Taiwan, R.O.C. on Jun. 6, 2024, the entire contents of which are hereby incorporated by reference.

This non-provisional application claims priority under 35 U.S.C. § 119(e) on U.S. provisional Patent Application No(s). 63/517,934 filed on Aug. 7, 2023 and U.S. provisional Patent Application No(s). 63/589,998 filed on Oct. 13, 2023, the entire contents of which are hereby incorporated by reference.

### BACKGROUND OF THE INVENTION

### 1. Field of the Invention

The present disclosure relates to endoscopic tube sets, and in particular to an endoscopic tube set comprising an endoscopic tube and a working element, the endoscopic tube having a position limiting component, and the working element having a cross section corresponding in shape to a position limiting opening of the position limiting component.

### 2. Description of the Related Art

Endoscopic tube sets are for use in observing, sampling and/or treating pulmonary lesions. Conventionally, an endoscopic tube set comprises an endoscopic tube and a working element (for example, an image capturing device, sampling device or rinsing device), allowing the working element to reach a lesion through a working channel in a catheter of the endoscopic tube.

However, the conventional endoscopic tube set in use is unable to prevent the working element from rotating in the working channel of the catheter and thus is likely to lead to lesion orientation misjudgment.

### BRIEF SUMMARY OF THE INVENTION

Conventional endoscopic tubes still have room for improvements. Therefore, it is an objective of the disclosure to provide an endoscopic tube set capable of preventing a working element from rotating in a working channel of a catheter and thereby precluding lesion orientation misjudgment.

To achieve the above and other objectives, the disclosure provides an endoscopic tube set comprising: an endoscopic tube comprising: a body; a catheter connected to the body, having therein one single working channel, having a proximal opening proximal to the body, and having a distal opening distal to the body; and a position limiting component fitted to the distal opening of the catheter, having a position limiting opening not round in shape, and having a position limiting mark; and a working element having a cross section corresponding in shape to the position limiting opening of the position limiting component and having at least one positioning mark.

In an embodiment of the disclosure, in the endoscopic tube set, at least a portion of the position limiting component is made of a material not penetrable by X-ray.

In an embodiment of the disclosure, in the endoscopic tube set, the position limiting component further has a plurality of guiding elements extending toward an inner edge of the position limiting component from the position limiting opening of the position limiting component.

In an embodiment of the disclosure, in the endoscopic tube set, the working element is an image capturing device, a sampling device or a rinsing device.

In an embodiment of the disclosure, the endoscopic tube in the endoscopic tube set further comprises an inflation device enclosing the catheter, having an expansion free end made of an elastic material and proximal to the distal opening of the catheter, having an end proximal to the distal opening of the catheter and closed, and having an end proximal to the proximal opening of the catheter and being in communication with a gas admitting component having a gas admitting opening for admitting an external fluid, wherein the expansion free end expands when a gas is introduced into a space between the inflation device and the catheter through the gas admitting opening.

In an embodiment of the disclosure, the endoscopic tube in the endoscopic tube set further comprises: a distal bending mechanism connected to the body and the catheter and comprising: an operating handle disposed outside the body and rotatably connected to the body; a spool disposed inside the body and connected to the operating handle; and a cable partially wound around the spool and having two ends connected to two sides proximal to the distal opening of the catheter respectively, wherein the operating handle can be rotated to drive the cable moving, causing a portion of the catheter to bend, with the bent portion being adjacent to the distal opening of the catheter.

In an embodiment of the disclosure, the endoscopic tube in the endoscopic tube set further comprises: a catheter rotating mechanism connected to the body and the catheter and comprising: a rotatable element connected to the catheter and rotatably connected to the body, wherein the rotatable element can be rotated to move the catheter, allowing the catheter to rotate.

In an embodiment of the disclosure, the endoscopic tube in the endoscopic tube set further comprises a sensing mechanism linked to the distal bending mechanism and the catheter rotating mechanism to sense a bending state of the catheter near the distal opening and a rotating state of the catheter.

In an embodiment of the disclosure, the catheter in the endoscopic tube set further has a rinsing port proximal to the proximal opening.

In an embodiment of the disclosure, the catheter in the endoscopic tube set further has a bellow cross parallel structure proximal to the distal opening.

The endoscopic tube set of the disclosure has advantages as follows: the endoscopic tube has a position limiting component, and the working element has a cross section corresponding in shape to a position limiting opening of the position limiting component, preventing the working element from rotating in a working channel of a catheter and thereby precluding lesion orientation misjudgment.

### BRIEF DESCRIPTION OF THE DRAWINGS

For the sake of clarity, the accompanying drawings are presented to show parts and components enlarged, omitted or schematically drawn. Furthermore, the dimensions of the parts and components shown in the accompanying drawings do not fully reflect their actual dimensions.

FIG. 1 is a schematic view of an endoscopic tube set according to an embodiment of the disclosure.

FIG. 2 is a schematic view of the endoscopic tube set viewed from another angle according to an embodiment of the disclosure.

FIG. 3 is an enlarged schematic view of a position limiting component of the endoscopic tube set according to an embodiment of the disclosure.

FIG. 4 is a schematic view of the position limiting component in an endoscopic tube of the endoscopic tube set according to an embodiment of the disclosure.

FIG. 5 is a cross-sectional view of an expansion free end of an inflation device of the endoscopic tube of the endoscopic tube set according to an embodiment of the disclosure.

FIG. 6 is an exploded view of the endoscopic tube of the endoscopic tube set according to an embodiment of the disclosure.

### DETAILED DESCRIPTION OF THE INVENTION

To facilitate understanding of the object, characteristics and effects of this present disclosure, embodiments together with the attached drawings for the detailed description of the present disclosure are provided.

### Embodiment of the Disclosure

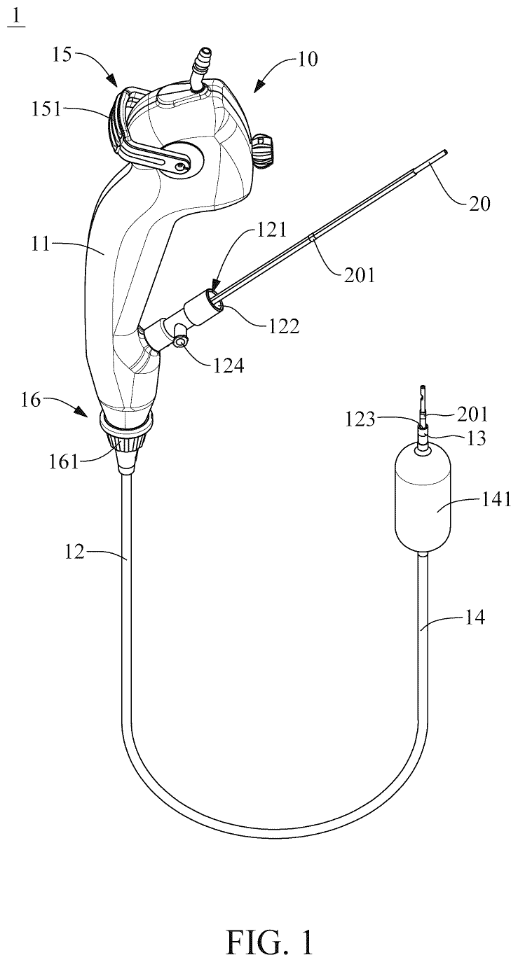

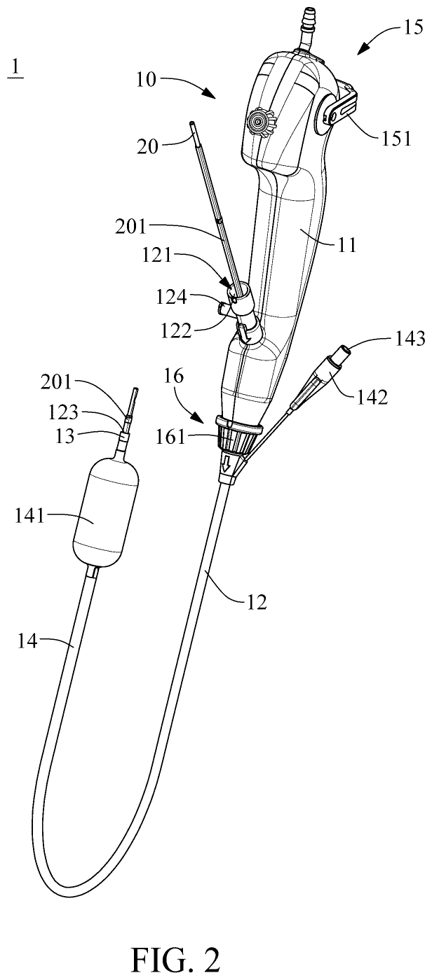

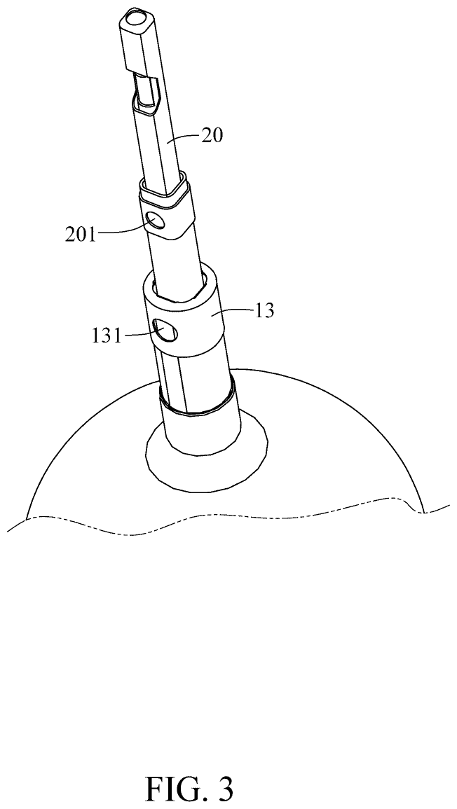

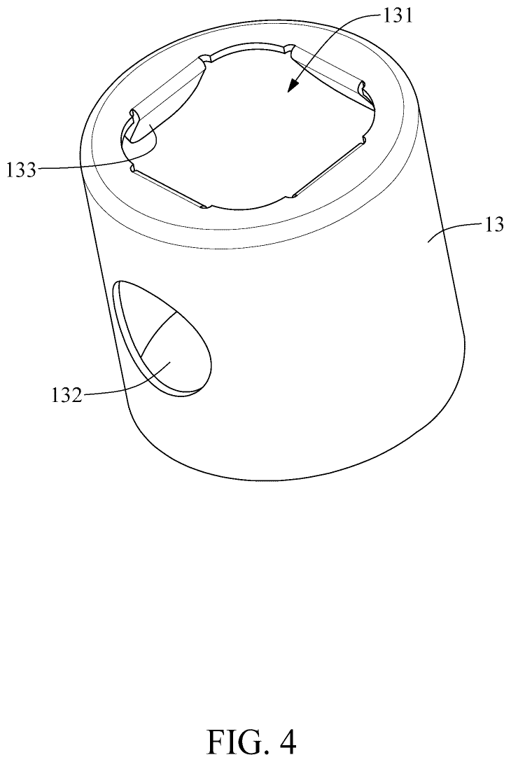

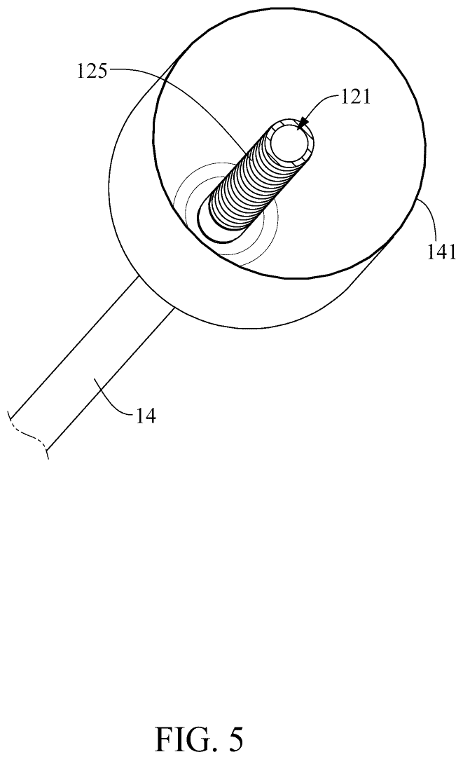

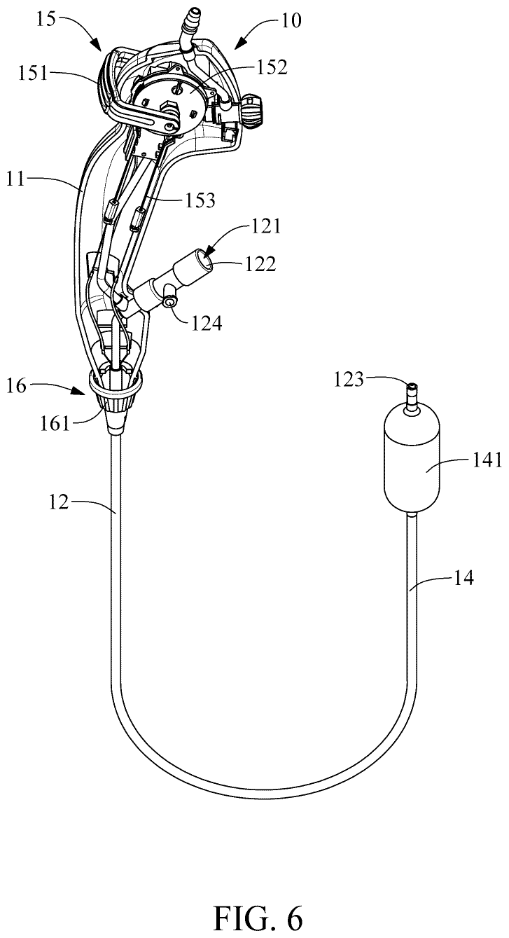

This embodiment provides an endoscopic tube set 1 shown in FIG. 1, FIG. 2, FIG. 3, FIG. 4, FIG. 5 and FIG. 6.

In this embodiment, the endoscopic tube set 1 comprises an endoscopic tube 10 and a working element 20.

In this embodiment, the endoscopic tube 10 of the endoscopic tube set 1 comprises a body 11. For example, the body 11 is made of plastic or metal, but the disclosure is not limited thereto.

In this embodiment, the endoscopic tube 10 further comprises a catheter 12 connected to the body 11, with the catheter 12 having therein one single working channel 121, having a proximal opening 122 proximal to the body 11, and having a distal opening 123 distal to the body 11.

In this embodiment, the endoscopic tube 10 further comprises a position limiting component 13 fitted to the distal opening 123 of the catheter 12, having a position limiting opening 131 not round in shape, and having a position limiting mark 132. In this embodiment, the position limiting opening 131 is square in shape, and the corners of the square are rounded, but the disclosure is not limited thereto; in a variant embodiment, the position limiting opening 131 can be in any shapes other than round shape. In this embodiment, the position limiting mark 132 is a hole disposed on a lateral side of the position limiting component 13, but the disclosure is not limited thereto. In this embodiment, the position limiting component 13 further has a plurality of guiding elements 133 extending toward the inner edge of the position limiting component 13 from the position limiting opening 131 of the position limiting component 13 to guide the working element 20 through the position limiting opening 131, but the disclosure is not limited thereto.

In this embodiment, the position limiting component 13 is made of metal, but the disclosure is not limited thereto. With metal not being penetrable by X-ray, the position limiting component 13 is imaged sharply by medical imaging technology based on X-ray, allowing users to identify the location of the position limiting component 13 in the human body.

In this embodiment, the working element 20 in the endoscopic tube set 1 has a cross section corresponding in shape to the position limiting opening 131 of the position limiting component 13 and has at least one positioning mark 201. The working element 20 is an image capturing device, a sampling device or a rinsing device, but the disclosure is not limited thereto.

For example, in this embodiment, the working element 20 is an image capturing device disposed in a working channel 121 of the endoscopic tube 10 and adapted to confirm that the distal opening 123 of the catheter 12 has already reached a position near the lesion. Then, the image capturing device is withdrawn from the working channel 121, and a sampling device or rinsing device functioning as the working element 20 is placed in the working channel 121 to sample and/or treat the lesion. In this embodiment, to render it easy and efficient to use and change the working element 20 of the endoscopic tube set 1, the cross section of the working element 20 corresponds in shape to the position limiting opening 131 of the position limiting component 13 to prevent the working element 20 from rotating in the working channel 121 of the catheter 12 and thereby preclude lesion orientation misjudgment. The positioning mark 201 of the working element 20 coordinates with the position limiting mark 132 of the position limiting component 13 to identify the positions of the endoscopic tube 10 and the working element 20 relative to each other. In a preferred embodiment, the working element 20 has one end proximal to the user and bearing a positioning mark 201, and thus the user can identify the positions of the endoscopic tube 10 and the working element 20 relative to each other even though the working element 20 has already been deep in the catheter 12.

In this embodiment, the endoscopic tube 10 of the endoscopic tube set 1 further comprises an inflation device 14 enclosing the catheter 12, but the disclosure is not limited thereto.

The inflation device 14 has an expansion free end 141 proximal to the distal opening 123 of the catheter 12. The expansion free end 141 is made of an elastic material. One end of the inflation device 14 is proximal to the distal opening 123 of the catheter 12 and is closed. One end of the inflation device 14 is proximal to the proximal opening 122 of the catheter 12 and is in communication with a gas admitting component 142 having a gas admitting opening 143 for admitting an external fluid.

An expansion free end 141 of the inflation device 14 is proximal to the distal opening 123 of the catheter 12 and is made of an elastic material. The expansion free end 141 expands (as shown in FIG. 1) when a gas is introduced into a space between the inflation device 14 and the catheter 12 through the gas admitting opening 143. When the gas is not introduced into a space between the inflation device 14 and the catheter 12, the expansion free end 141 is attached to the catheter 12 in order to penetrate the lung deep.

In the embodiment where the endoscopic tube 10 of the endoscopic tube set 1 further comprises the inflation device 14, the expansion free end 141 expands to fix the distal opening 123 in place when the image capturing device functioning as the working element 20 confirms that the distal opening 123 of the catheter 12 has already reached a position near the lesion. Therefore, the positions of the distal opening 123 and the lesion relative to each other are fixed to prevent the distal opening 123 from undergoing displacement relative to the lesion while the working element 20 is being changed.

In this embodiment, the endoscopic tube 10 of the endoscopic tube set 1 further comprises a distal bending mechanism 15 connected to the body 11 and the catheter 12, but the disclosure is not limited thereto.

The distal bending mechanism 15 comprises: an operating handle 151 disposed outside the body 11 and rotatably connected to the body 11; a spool 152 disposed inside the body 11 and connected to the operating handle 151; and a cable 153 partially wound around the spool 152 and having two ends connected to two sides proximal to the distal opening 123 of the catheter 12 respectively. Therefore, the operating handle 151 can be rotated to drive the cable 153 moving, causing a portion of the catheter 12 to bend, with the bent portion being adjacent to the distal opening 123 of the catheter 12.

In this embodiment, the endoscopic tube 10 of the endoscopic tube set 1 further comprises a catheter rotating mechanism 16 connected to the body 11 and the catheter 12, but the disclosure is not limited thereto.

The catheter rotating mechanism 16 comprises a rotatable element 161 connected to the catheter 12 and rotatably connected to the body 11. Therefore, the rotatable element 161 can be rotated to move the catheter 12, allowing the catheter 12 to rotate.

The distal bending mechanism 15 and/or the catheter rotating mechanism 16 enables users to precisely control the orientation of the distal opening 123 of the catheter 12, allowing the distal opening 123 of the catheter 12 to reach any lesion deep in the lung.

In this embodiment, the endoscopic tube 10 of the endoscopic tube set 1 further comprises a sensing mechanism (not shown) linked to the distal bending mechanism 15 and the catheter rotating mechanism 16 to sense the bending state of the catheter 12 near the distal opening 123 and the rotating state of the catheter 12, but the disclosure is not limited thereto.

The sensing mechanism enables users to monitor the bending state of the catheter 12 near the distal opening 123 and the rotating state of the catheter 12 in real time, allowing the distal opening 123 of the catheter 12 to reach any lesion deep in the lung.

In this embodiment, in the endoscopic tube 10, the catheter 12 further has a rinsing port 124 proximal to the proximal opening 122, but the disclosure is not limited thereto. Owing to the rinsing port 124, the endoscopic tube 10 rinses a lesion while the working element 20 is staying in the working channel 121, further enhancing the flexibility of the application of the endoscopic tube 10.

In this embodiment, in the endoscopic tube 10, the catheter 12 has a bellow cross parallel structure 125 proximal to the distal opening 123, but the disclosure is not limited thereto. Owing to the bellow cross parallel structure 125, a portion of the catheter 12 is near the distal opening 123 and has sufficient rigidity and bending capability, allowing the distal opening 123 of the catheter 12 to reach any lesion deep in the lung.

The invention is disclosed above by preferred embodiments. However, persons skilled in the art should understand that the embodiments are illustrative of the invention only, but shall not be interpreted as restrictive of the scope of the invention. Please note that all variations and replacements equivalent to the embodiments shall be deemed falling within the scope of the invention. Therefore, the legal protection for the invention shall be defined by the appended claims.

While the present disclosure has been described by means of specific embodiments, numerous modifications and variations could be made thereto by those skilled in the art without departing from the scope and spirit of the present disclosure set forth in the claims.

What is claimed is:

1. An endoscopic tube set, comprising:
an endoscopic tube, comprising:
a body;
a catheter connected to the body, having therein one single working channel, having a proximal opening proximal to the body, and having a distal opening distal to the body; and
a position limiting component fitted to the distal opening of the catheter, having a position limiting opening not round in shape, and having a position limiting mark; and
a working element having a cross section corresponding in shape to the position limiting opening of the position limiting component and having at least one positioning mark.

2. The endoscopic tube set of claim 1, wherein at least a portion of the position limiting component is made of a material not penetrable by X-ray.

3. The endoscopic tube set of claim 1, wherein the position limiting component further has a plurality of guiding elements extending toward an inner edge of the position limiting component from the position limiting opening of the position limiting component.

4. The endoscopic tube set of claim 1, wherein the working element is an image capturing device, a sampling device or a rinsing device.

5. The endoscopic tube set of claim 1, wherein the endoscopic tube further comprises an inflation device enclosing the catheter, having an expansion free end made of an elastic material and proximal to the distal opening of the catheter, having an end proximal to the distal opening of the catheter and closed, and having an end proximal to the proximal opening of the catheter and being in communication with a gas admitting component having a gas admitting opening for admitting an external fluid, wherein the expansion free end expands when a gas is introduced into a space between the inflation device and the catheter through the gas admitting opening.

6. The endoscopic tube set of claim 1, wherein the endoscopic tube further comprises:
a distal bending mechanism connected to the body and the catheter and comprising:
an operating handle disposed outside the body and rotatably connected to the body;
a spool disposed inside the body and connected to the operating handle; and
a cable partially wound around the spool and having two ends connected to two sides proximal to the distal opening of the catheter respectively,
wherein the operating handle can be rotated to drive the cable moving, causing a portion of the catheter to bend, with the bent portion being adjacent to the distal opening of the catheter.

7. The endoscopic tube set of claim 6, wherein the endoscopic tube further comprises:
a catheter rotating mechanism connected to the body and the catheter and comprising:
a rotatable element connected to the catheter and rotatably connected to the body,
wherein the rotatable element can be rotated to move the catheter, allowing the catheter to rotate.

8. The endoscopic tube set of claim 7, wherein the endoscopic tube further comprises a sensing mechanism linked to the distal bending mechanism and the catheter rotating mechanism to sense a bending state of the catheter near the distal opening and a rotating state of the catheter.

9. The endoscopic tube set of claim 1, wherein the catheter further has a rinsing port proximal to the proximal opening.

10. The endoscopic tube set of claim 1, wherein the catheter further has a bellow cross parallel structure proximal to the distal opening.
